(19) United States
(12) Patent Application Publication
(10) Pub. No.: US 2011/0165671 A1
(43) Pub. Date: Jul. 7, 2011

(54) PROCESS FOR PROVIDING AN ASSEMBLY OF CELL MICROCARRIERS

(75) Inventors: David Halter, Utrecht (NL); Emiel Peeters, Eindhoven (NL); Roel Penterman, Eindhoven (NL); Ralph Kurt, Eindhoven (NL); Dirk Jan Broer, Geldrop (NL)

(73) Assignee: KONINKLIJKE PHILIPS ELECTRONICS N.V., EINDHOVEN (NL)

(21) Appl. No.: 13/002,991

(22) PCT Filed: Jun. 26, 2009
(86) PCT No.: PCT/IB2009/052757

§ 371 Date: Mar. 16, 2011

(30) Foreign Application Priority Data
Jul. 14, 2008 (EP) 08104740.9

(51) Int. Cl. C12N 5/071; C12M 3/00; B28B 1/14

(52) U.S. Cl. 435/325; 435/307.1; 264/299

(57) ABSTRACT

The present invention is related to a process for providing an assembly of cell microcarriers, comprising the steps of providing planar, two-dimensional objects having two sides (“flakes”), wherein these objects comprise a material which, upon application of an extrinsic stimulus, is transferred from the planar state into a rolled state, providing cells on one side of said flakes (“cell-bearing side”), transferring the flakes from the planar state into a rolled state (“cell wrap”) by application of said extrinsic stimulus, and coupling at least one type of binding agent to the flakes.

### FIELD OF THE INVENTION

The present invention is related to a process for providing an assembly of cell microcarriers. The said process is related to the treating of cultured cells, to cell handling, cell delivery and/or cell targeting in tissue and/or organ engineering, and cell therapy applications.

### BACKGROUND OF THE INVENTION

Regenerative medicine is a new upcoming discipline within the field of medical sciences. There are numerous methods and approaches used in regenerative medicine.

In tissue engineering in vitro, tissue is grown outside the body utilising scaffolds and cells. The engineered tissue is subsequently implanted in a patient in order to replace damaged or lost tissue.

In tissue engineering in vivo, scaffolds are placed in damaged tissue areas with the aim of inducing growth of cells from the surrounding healthy tissue to restore damaged tissue.

Cell therapies are based on the delivery of cells, particularly stem cells, to a damaged tissue area in order to restore the tissue function.

There are therapies utilizing growth factors, e.g. cytokines and chemokines, in order to recruit endogenous cells to the damaged tissue area. The growth factors can be delivered directly to the area of interest, e.g. via injection.

Cells play a crucial role in both in vitro tissue engineering and cell therapies where the cells are first harvested from the appropriate cell source, i.e. from the patient (“autologous”) or from a donor (alleogenic) and subsequently subjected to several different steps until they are finally introduced again in the patient to replace or restore damaged tissue. Despite the tremendous progress in cell therapies and tissue engineering over the last few years, basic and essential steps, e.g. cell handling, cell (in) growth in scaffolds, cell differentiation, cell delivery and cell retainment are still problematic and need further improvement before regenerative medicine becomes clinically relevant.

In EP07101104 the problem of cell handling and cell delivery is addressed by wrapping cells into multilayer flakes comprising a hydrogel. Initially, cells are grown on planar flakes where they are exposed to the culture environment for optimum growth, while cell handling and cell delivery the planar flakes are transferred into the rolled state (termed “microcarriers” or “cell wraps” herein).

The said flakes offer an efficient way for wrapping cells and protecting them from the environment. During cell growth the flakes are attached to a surface. The flakes can be single layers with build-in stress, bi-layers or multi-layers. When transferred into the rolled state, the flakes will wrap the cells and thus create the cell wraps which are as well on subject of the present invention.

However, the said approach does not provide a solution for the specific delivery or targeting of the cell wraps to the appropriate targeting tissue. This is however crucial as cells delivered to the wrong place within the body might lead to uncontrolled growth of these cells in undesired places.

Moreover, it turned out that adhesion of the cell wraps to the target tissue, and adhesion among similar and different cell wraps, is a serious problem. Finally, it has been shown that the design of tissues and organs, or the repair of damaged tissues and organs, is a complicated matter which requires a high degree of control, both in terms of the spatial arrangement of the cells and/or cell wraps, as well as the chronological order of the cell and/or cell wrap binding process, particularly when it comes to the design and/or repair of highly complex tissues and/or organs. The approach as set forth above does however not provide any solutions to meet these demands.

### SUMMARY OF THE INVENTION

It is the object of the present invention to provide a process, an object, and an assembly, which overcomes the above mentioned shortcomings.

This object is achieved by the method and the two-dimensional object as set forth under the independent claims. The dependent claims indicate preferred embodiments. In this context it is noteworthy to mention that all ranges given in the following are to be understood as that they include the values defining these ranges.

In accordance with the invention, a process for providing an assembly of cell microcarriers is provided, which comprises the steps of
a) providing planar, two-dimensional objects having two sides (“flakes”), wherein these objects comprise a material which, upon application of an extrinsic stimulus, is transferred from the planar state into a rolled state,
b) providing cells on one side of said flakes (“cell-bearing side”),
c) transferring the flakes from the planar state into a rolled state (“cell wrap”) by application of said extrinsic stimulus, and
d) coupling at least one type of binding agent to the flakes before or after any of steps a)-c).

By doing so, at least one of the following advantages can be reached for a wide range of applications within the present invention:
By the “cell wrapping” technique it is possible to shield cells; e.g. during storage.
The “flakes” allow to build up complex structures; as will be described later on.
Due to the extrinsic stimulus it is possible to separate the steps of providing the cells on the flakes and the “wrapping” step, rather than being forced to grow and/or provide cells in a topological unfavourable environment.

Said step of coupling at least one type of binding agent to the flakes can take place both in their planar state (i.e. before step c) as well as in their rolled state (i.e. after step c). Furthermore, the said step can take place before the cells are attached to the flakes (i.e. before step b), as well as after the cells are attached to the flakes (i.e. after step b).

The term “coupling at least one type of binding agent to the flakes”, as used herein, refers to
(i) a literal attachment of separate entities having binding agent capabilities to the flakes, e.g. by binding them covalently or non-covalently to the flakes, and/or
(ii) a modification of compound matter which already forms part of the flakes, i.e. by chemical functionalization or activation of surface molecules of the flakes.

As regards option (i), a preferred embodiment does involve the use of crosslinkers. Crosslinkers are molecules which can establish a covalent bond between one another. Homobifunctional crosslinkers have two identical reactive groups, while Heterobifunctional crosslinkers possess two different reactive groups that allow for sequential (two-stage) conjugations. Crosslinkers contain at least two reactive groups. Target groups for crosslinking include primary amines, sulfhydryls, carbonyls, carbohydrates and carboxylic acids (Table 1).

TABLE 1

| Reactive Group | Targets |
|---|---|
| Aryl Azide | Non-selective |
| Carbodiimide | Amine/Carboxyl |
| Hydrazide | Carbohydrate |
| | (oxidized) |
| Hydroxymethyl | Amine |
| Phosphine | |
| Imidoester | Amine |
| Isocyanate | Hydroxyl (non- |
| | aqueous) |
| Maleimide | Sulfhydryl |
| NHS-ester | Amine |
| PFP-ester | Amine |
| Psoralen | Thymine |
| Pyridyl Disulfide | Sulfhydryl |
| Vinyl Sulfone | Sulfhydryl, Amine, |
| | Hydroxyl |

An example for crosslinking in the above sense is the coupling of protein-based binding agents (e.g. antibodies, or biotin, or the like) to the flakes with EDC/NHS-chemistry, i.e. N-(3-dimethylaminopropyl)-N′-ethylcarbodiimide (EDC) and N-hydroxysuccinimide (NHS).

Other possibilities comprise the coupling of binding agents be means of ligation reactions (particularly in case the binding agents are nucleic acids, see below), by reactions of epoxides (which form part of the flakes) with amines, by Michaels additions of amines to double bonds or by non-covalent attachment, e.g. multiple hydrogen bonding, oligonucletide hybridization, and/or methyl-ligand complexation.

As regards option (ii) it is possible incorporate the binding agent during the formation process of the flakes, e.g. by modifying the binding agent with a reactive groups and mix it with a hydrogel monomer mixture before polymerization of the latter.

In a preferred embodiment, the process according to the invention is characterized in that said binding agents are coupled to the side of the flakes which is opposite to the cell-bearing side. The said process thus provides cell-containing rolled flakes, also termed “cell wraps” in the following, which bear, on their outer side, binding agents capable of building up bonds to other entities.

In yet another preferred embodiment of the invention it is provided that said process comprises the additional step of
e) binding at least one cell wrap thus achieved to at least one other entity by means of at least one of said binding agents.

Such other entities include
(i) other cell wraps,
(ii) non-cellular compounds to which the cell wraps are to be bound, e.g. three dimensional matrices, like solid porous scaffolds which are being used for in vitro tissue engineering, for example comprising biodegradable matter and/or collagen,
(iii) living matter, e.g. extracellular matrix, cells, tissues, and/or organs,
(iv) circulating materials, e.g. viruses, antibodies, bacteria, spores, and/or
(v) surgical instruments, implants, culture dishes, and/or patterned surfaces of culture dishes.
or any other entities to which binding of the said cell wraps might be desirable.

It is crucial that these other entities bear at least
(i) one complementary binding agent, or
(ii) comprise a moiety that is complementary to the said binding agent (“intrinsic binding moiety”).

The term “complementary binding agent”, as used herein, shall refer to a binding agent which is capable of building up a bond with another binding agent. It is preferred that the said complementary binding agent binds with high specifity to the latter. By this means, assemblies between the said cell wraps and the respective other entities can be built op. If different pairs of binding agent and complementary binding agent are being used, assemblies of high complexity can thus be produced.

Furthermore, a self-assembling complex can thus be produced, namely by merely coupling different entities to the said cell wraps.

Option (i) is a preferred option, while option (ii) may be used in some cases where the second entity has an intrinsic moiety that is complementary to the said binding agent. Such moiety may for example be an antigen (in this case the binding agent is an antibody), a tissue specific marker (in this case the binding agent may be a protein that recognizes said marker) or a sugar (in this case the binding agent may be a lectin, for example).

It is important to mention that antigens, tissue specific markers and sugars as well act as complementary binding agents, namely when they are not integral part or intrinsic moieties of the said other entity.

This means that whether a binding agent qualifies as a binding agent in broad sense or as an intrinsic binding moiety in the above sense is a question of whether it is integral part or intrinsic moiety of the said entity.

In a preferred embodiment, more than one type of binding agents can be added to a cell wrap. For example one binding agent type may be selected in such way that it binds to a three dimensional matrix, whereas another binding agent is selected in such way that it may bind other cell wraps, carrying complementary binding agents. According to this embodiment, the first cell warp type may be used as an anchoring device, to which other cell wrap types may then bind.

The recited binding agents can accomplish either covalent bonds or non-covalent bonds.

In a preferred embodiment of the invention it is provided that that said binding agents are selected from the group consisting of proteins and polypeptides, nucleic acids, molecular tags, ligands, magnetic entities and/or charged groups.

It is noteworthy that the above mentioned binding agents are capable of building up non-covalent bonds with other binding agents.

The said non-covalent bonds comprise, among others, hydrogen bonds, protein protein interactions, ionic bonds, hydrophobic interactions, Van der Waals forces, and Dipole-dipole bonds.

It is a well known fact that there is a vast number of proteins and oligopeptides that exhibit specific binding properties towards a given target. Non limiting examples for these proteins are, among others, antibodies (particularly monoclonal antibodies), collagen binding proteins, ankyrin repeats, integrins, streptavidin, avidin and biotin,

Collagen binding proteins are a proteins that bind to collagen. Examples for such protein are, for example CbpA, a collagen-binding protein of A. pyogenes, CNE, a collagen-binding protein of Streptococcus equi, KINDLIN-3, Collagen binding protein Mip of Legionella pneumophila, type I-IV collagen-binding proteins, integrin proteins and so forth.

Examples for collagen binding proteins are given in Svensson L, Oldberg A, Heinegard D. Collagen binding proteins. Osteoarthritis Cartilage 2001; 9:S23-8. 4, the content of which is herein incorporated by reference.

The terms “nucleic acid”, “polynucleotide” and “oligonucleotide” as used herein, refer to, among others, monomers, oligomers and polymers of RNA, DNA, LNA, PNA, Morpholino and other nucleic acid analogues. A peptide nucleic acid (PNA) is an artificially synthesized polymer similar to DNA or RNA which cofeatures backbone composed of repeating N-(2-aminoethyl)-glycine units linked by peptide bonds. A locked nucleic acid (LNA) is a modified RNA nucleotide in which the ribose moiety is modified with an extra bridge connecting the 2′ and 4′ carbons. Morpholinos are synthetic analogues of DNA which differ structurally from DNA in that while Morpholinos have standard nucleic acid bases, those bases are bound to morpholine rings instead of deoxyribose rings and linked through phosphorodiamidate groups instead of phosphates.

The said oligonucleotides can be single-stranded or, at least in part, double stranded. In the latter case, for carrying out a binding between a binding agent and its complementary binding agent, the oligonucleotides may either have so called “sticky ends”, which are characterized by single strand overhangs. An overhang is a stretch of unpaired nucleotides in the end of a DNA molecule. These unpaired nucleotides can be in either strand, creating either 3′ or 5′ overhangs. Such sticky ends may for example look as follows:

5′-ATCTGACTATTTCG-3′
3′-TAGACTGA-5′

The said oligonucleotide would be complementary to another sticky end-oligonucleotide of the following sequence:

5′-ATCTGACTCGAAAT-3′
3′-TAGACTGA-5′

Another option when using double stranded oligonucleotides as binding agents is to heat up the cell wraps to a temperature above the melting temperature of the double-stranded oligonucleotide, in order to achieve double strand denaturation. This is commonly done by heating the mixture to a temperature of above 85° C.

One way to determine the said melting temperature is the so-called Wallace method, which is suitable for oligonucleotides less than 18mers in length. It is being done by counting the frequency of each nucleotide base. The reasoning behind the method is that, because cytosine-guanine pairs form three hydrogen bonds compared to the two hydrogen bonds between adenosine and thymine, they contribute more to the stability of a double-helix

Tₘ=2(A+T)+3(G+C)

The said approach is highly beneficial as it allows for a controlled coupling of cell wraps equipped with double stranded oligonucleotide binding agents, simply by enhancing the temperature to a point above melting temperature.

The binding process of different cell wraps can thus simply be switched on and off. This approach is of course only possible if the cells allow such procedure, i.e. if they are heat resistant. However, agents are available which are capable of lowering the nucleotide melting temperature, e.g. the disaccharide Trehalose.

These agents help to increase the compatibility of the said approach with living cells.

Another way to use this approach without impairing the cells comprised in the cell wraps is the application of localized heating techniques, like ultrasound, in particular high focus ultrasound (HIFU) or light (e.g. two photon infrared excitation, photonic needles, and the like), or the use of magnetic particles which are set under high frequency vibrations by means of a focused alternating electromagnetic field, thus creating a locally focused temperature increase.

The term “ligand”, as used herein, refers to a substance that is able to bind to and form a complex with a molecule to serve a biological purpose, e.g. to carry out a cell signalling process, or the like.

The term “magnetic entities”, as used herein, refers to entities which have diamagnetic, paramagnetic, ferromagnetic, ferrimagnetic and/or superparamagnetic properties, in such way that they exert attractive or repulsive forces on other materials. The said magnetic entities may for example adopt the form of tethered magnetic beads.

The term “charged group”, as used herein, refers to a substance that bears negative, positive and/or neutral electric charges, including partial charges. These groups are capable of building up an ionic bond between the cell wrap an another entity, e.g. another cell wrap bearing a complementary charge. Examples for such charged groups which may be coupled to the flakes may be selected from the group consisting of, among others, charged amino acids (aspartic acid, glutamic acid, arginine, hististine, lysine), organic acids and bases, sulphonate groups, and/or pyridine groups.

The terms “complementary nucleic acid” and “complementary oligonucleotide” refer to nucleic acids, polynucleotides and/or oligonucleotides which have base sequence comprising any of the bases cytosine (C), guanine (G), adenine (A), thymine (T) and uracil (U), or to Hypoxanthine, Xanthine, 7-Methylguanine, 5,6-Dihydrouracil, 5-Methylcytosine, isoguanine and isocytosine, that is capable of hybridizing to another nucleic acid, polynucleotide and/or oligonucleotide according to the Watson-Crick base pairing mechanism.

The terms “antibody” and “monoclonal antibody” refer to immunoglobulin molecules exhibiting a binding affinity towards a given antigen, and which are either of produced by immunized mammals or by recombinant microorganisms.

Lectins are sugar-binding proteins which are highly specific for their sugar moieties. They typically play a role in biological recognition phenomena involving cells and proteins. For example, some bacteria use lectins to attach themselves to the cells of the host organism during infection.

Ankyrin repeats are derived from natural ankyrin repeat proteins which are used in nature as versatile binding proteins with diverse functions such as cell signalling, kinase inhibition or receptor binding just to name a few. These ankyrin repeats are for example described in EP1332209.

Streptavidin is a 53 kD protein purified from the bacterium Streptomyces avidinii, which exhibits strong affinity for the vitamin biotin; the dissociation constant (K_{d}) of the biotin-streptavidin complex is on the order of ˜10-15 mol/l. Avidin is a similar protein which has as well a strong affinity to biotin.

The term “molecular tag” (sometimes also termed “affinity tag”) refers to molecules which are, for example, being used for the purification of proteins. These tags comprise, among others,
Immobilized metal ions, like Ni-NTA
His-Tag (Hexahistidine)
chitin binding protein (CBP)
maltose binding protein (MBP)
rProtein L
Cκ domain
Flag-Tag (DYKDDDDK)
Strep-Tag
Arg-Tag
HA-tag
myc-tag
GST (Glutathion-S-Transferase)
V5-tag
BCCP tag
Calmodulin-tag
S-tag
GFP-tag (green fluorescent protein)
Protein A-tag

The person skilled in the art will find in the above listing a comprehensive teaching which enables him, without the requirement of additional inventive step, to find
(i) binding agents complementary to the above molecular tags, and/or
(ii) other molecular tags not mentioned here.
For the said purpose, the skilled person may refer to respective textbooks, literature databases, catalogues and the like.

As already apparent from the above, a binding agent may have a complementary binding agent to which it my bind in order to link one flake according to the invention to another. Table 2 gives an overview over some preferred pairs of binding agents.

TABLE 2

| Binding agent 1 | Binding agent 2 (complementary) |
|---|---|
| HisTag | metal ions (e.g. Ni-NTA) |
| antibody | Antigen* |
| lectin | sugar, glycoproteins, glycolipds* |
| biotin | streptavidin, avidin |
| collagen binding proteins | Collagen* |
| oligonucleotide | complementary oligonucleotide |
| tissue specific ligand | tissue specific receptor* |
| Magnetic beads | Magnetic beads of complementary polarity |
| charged group (e.g. “+”) | complementarily charged group (e.g. “−”) |
| hydrophilic group | hydrophilic group* |
| hydrophobic group | hydrophobic group* |

Some of the complementary binding agents (particularly those marked with an asterisk*) may as well serve as intrinsic binding moieties, depending on whether they are integral part or intrinsic moiety of the respective entity.

In yet another preferred embodiment of the present invention it is provided that said binding agents are selected in such way that they confer, to the flakes, or to subsections of the former, hydrophobic and/or hydrophilic properties.

In this preferred embodiment, the term “binding agent” is not to be understood as to build up a covalent or non-covalent bond to another entity.

If, for example a flake is provided with an alternating pattern of hydrophobic and hydrophilic binding agents, different cell wraps thus produced will, when mixed, self-assemble in an orderly manner, in such way that hydrophilic regions of different cell wraps are in contact with each other and hydrophobic regions of different cell wraps are in contact with each other as well (see, for example, FIG. 6).

In yet another preferred embodiment of the present invention it is provided that said binding agents are capable of building up covalent bonds.

In a preferred embodiment these covalent bonds are bio-orthogonal, i.e.
(i) they must not have detrimental effects on the survival of the cells grown on the flakes (the must be biocompatible),
(ii) they need to have a selective reactivity in order to provide a specific binding behavior, and
(iii) they must not have detrimental effects on the survival and function of the tissue or body in which the reaction takes place

Binding mechanisms capable of building up such bonds comprise, for example, the so called “Staudinger reaction” (i.e. the combination of an azide with a phosphine or phosphate to produce an iminophosphorane), the “Staudinger ligation” (i.e. formation of an iminophosphorane through nucleophilic addition of the phosphine at the terminal nitrogen atom of the azide and expulsion of nitrogen), or the so called “click reaction” (i.e. so called “Strain Promoted [3+2] Azide-Alkyne cycloaddition”).

The said binding mechanisms and binding agents capable of carrying out such mechanism are for example disclosed in US20080075661A1, WO2007110811A2 and WO2007039864.

In another preferred embodiment of the present invention it is provided that at least two different types of binding agents are added to said flakes in a patterned fashion.

In this context, the term “patterned fashion” means that members of the different binding agents are segregated from one another in such fashion that given sections of the flakes comprise only a single type of binding agent. The patterns thus obtained may for example be regular patterns, e.g. a grating, an array of stripes, a grid, a two dimensional array of spots or circles, and the like. This definition does moreover include heterogeneous patterns and irregular patterns.

The said patterns can for example be obtained by microprinting (e.g. microcontact printing, inkjet printing) or lithographic and/or photolithography techniques.

Furthermore, it is provided in a preferred embodiment of the present invention that step b) comprises the substeps of
b1) seeding cells on said flakes, and
b2) growing said cells.

After said transfer the once planar flakes may for example adopt a cylindrical shape with open or substantially closed ends. See FIG. 2 or 3 for examples of such shape. Typical dimensions of the flakes are in the order of the dimensions of the cells or somewhat bigger than that. This means that, according to a preferred embodiment of the present invention, the size or the length of the planar hydrogel flakes is ≧10 μm and ≦100 mm, more preferably ≧10 μm and ≦10 mm, and more preferably ≧20 μm and ≦1 mm, and most preferably ≧50 μm and ≦500 μm.

According to another preferred embodiment of the present invention, the thickness of the planar flakes is ≧100 nm and ≦1 mm, more preferably ≧500 nm and ≦500 μm, and most preferably ≧1 μm and ≦100 μm.

According to yet another embodiment of the present invention, the inner diameter of the flakes in the rolled up state (comprising cells) is ≧1 μm and ≦5 mm, more preferably ≧5 μm and ≦500 μm, and most preferably ≧10 μm and ≦100 μm.

In addition to mere cell growth, the cells can remain on the planar flakes for differentiation before the flakes are transferred into the rolled state. This means that, between steps b) and c), a differentiation step can be introduced. Likewise, cell division, cell growth, and cell profilation can be promoted between steps b) and c). The person skilled in the art may readily select from his knowledge, or from appropriate references, the conditions which are to be applied in order to achieve cell differentiation as referred to above.

In most cases, standard cell culture conditions will be used. Mammal cells, including human cells, for example, are preferably cultured at 37° C. and under a 5% CO₂-atmosphere in order the keep the pH in a physiological range. Standard growing media, such as synthetic media complemented with FCS (Fetal calf serum) may be used, and growth factors, antibiotics and the like may be added if necessary. Insect cells, plant cells and prokaryotic cells, which also fall under the scope of the present invention, will however be treated with different conditions, which are well known per se from the state of the art.

It is moreover understood that the shape of the flakes can resemble a square, a rectangle or a parallelogram. More complex shapes can however be used in order to achieve a more complex wrapped state. For example, in order to achieve a helix-like wrapped state, the flakes should have a shape which resembles an elongated parallelogram. Likewise, flakes can as well have a circular, elliptic, trapezium like, hexagonal, polygonal or triangular shape, which in each case will lead to different wrapped states. A surface structured topologically, mechanically, or in composition, and a more complex layering within the flakes can also contribute to achieve a more complex wrapped state.

In this context, it is worth mentioning that the rolled up state can include two cases, namely at least partially multilayered cylinder, as can be seen in FIG. 2, or cylindrical body just substantially closed. The latter means that there is basically no overlap of the two touching edges, which would also allow sharper angles between the closing sides of the flake.

Usually, it desired that the cells are only present on the flakes and not on the substrate in between the flakes. In order to avoid that cells settle down in the interstitium between the flakes, the surface of the substrate underneath can be modified such that cells do not adhere to it, or that the cells have a clear preference to grow on the flakes rather than on the substrate. However, in some cases it does not matter if cells also grow on the substrate. As soon as the flakes are released and rolled up, the substrate with left-over cells can be discarded.

In some special cases it can even be beneficial to have cells in the interstitium between the flakes. For instance, some difficult cell-types need co-feeder cells for growth, namely for the production of the appropriate growth factors. These feeder cells can be seeded in between the flakes, while the cells of interest are grown on the flakes.

In yet another embodiment, not only various cell wraps can be attached to each other, but also containers or cell wraps containing containers could be attached to cell wraps. These containers can e.g. be used to deliver growth factors which help to control the cells (e.g. growth factors that control the differentiation of cells). The term “containers” does also comprise hydrogel articles comprising the said substances, and which are used for controlled release of these substances. Said release can as well be induced by any of the external stimuli mentioned herein. The big versatility described above could be used to control the spatial and temporal release of different growth factors.

In many cases to get a proper differentiation, several growth factors/cytokines have to be delivered in a specific order at precisely defined times. With the present embodiment, a first set of cell wraps with cells could be delivered, followed by a cell wrap containing a growth factor “A”. After a certain time the container with growth factor “A” could be removed again and another container with growth factor “B” could be delivered. Different growth factors could therefore work in a concerted action on the cell delivered in the first place. After that another set of cells could for example be delivered and treated in a similar or different way.

According to a preferred embodiment of the inventive process, said stimulus is selected from the group consisting of
induced change of pH,
induced change of temperature,
induced exposure to electromagnetic waves,
induced exposure to ions, specific salts or organic compounds, or to a given concentration thereof,
application of an electric field,
application of a magnetic field,
application of sound,
application of vibrations,
induced exposure to, or an induced suppression of, enzymes and other biomolecules,
induced release from the substrate, and/or
induced exposure to a solvent composition.

As regards a thermal stimulus, it is for example preferable that upon heating (e.g. ≧36° C.) the flakes are in the planar state, whereas they are transferred into a rolled state upon cooling (e.g. ≦35° C.). This is especially beneficial, as by cooling the flakes (and the cells comprised therein) cells will stop growing, and their metabolic rate is reduced. Cells can thus be stored for transport or prepared for the respective application, without injury or detrimental effects. It is as well preferred that the stimuli applied are selected as not to deteriorate the physiology of the cells.

Moreover, in a preferred embodiment it is provided that the flakes comprise a stimulus responsive material which has reversible swelling properties, or the like, i.e. the said stimulus can be applied many times.

It is understood that the term “electromagnetic waves” includes visible light, ultraviolet and infrared light, X-ray, microwaves, radiowaves and the like. It is further understood that the term “sound” includes ultra- and infrasound, as well as audible sound.

An induced release from the substrate, which may result in transfer of the flakes into the rolled state, can for example be accomplished by a temperature shift and/or a pH shift.

It is to be understood that it can as well be provided that flakes, in their rolled state, can be responsive to external stimuli of the above kind, and can thus be transferred into the planar state again when the right stimulus is provided, thus exposing the cells to the environment. For this purpose, it can for example be provided that the flakes are transferred into the rolled state by decreasing, or increasing, the pH, or temperature, and transferred into the planar state by increasing, or decreasing, the pH, or temperature, again.

Before or during transferring the flakes into the rolled state, it is preferred that they are being released from a substrate they adhere to. The release and the rolling of the flakes can, in a preferred embodiment, both be initiated by the swelling of the flakes. In this embodiment, the release and transfer into the rolled state happen at the same time. In the unswollen state there are little to no stresses in the flakes, and although the adherence of the flakes to the substrate is not optimal the flakes will adhere to the substrate. In the swollen state the built-up stress and change in hydrophilicity of the hydrogel layer will cause release of the flakes. The cultured cells include stem cells and differentiated cells, e.g. adult mesenchymal stem cells, adult hemopoietic stem cells, adipose derived adult stem cells, embryonic stem cells, chrondrocytes, osteoblasts, osteocytes, myoblasts, cardiac myocytes, fibroblasts, B cells, T cells, dendritic cells, erythrocytes, lymphoid progenitor cells, myeloid progenitor cells, etc, of both human and non-human origin. However, for research purposes, or for the production of cells which are later being used in the production of biological matter, even immortalized cells can be used, i.e. hybridoma cells and the like.

This means as well that in case omnipotent stem cells are being used, step c) will take place before the cells start to differentiate. In case pluripotent cells are being used, a differentiation (at least in part) may take place before step c) is induced, as illustrated above.

In a further preferred embodiment the flakes comprise labels or markers. Such labelling can for example be accomplished by use of dyes, magnetic beads, X-Ray markers, MRI-markers or targeting moieties like antigens, lectins, reactive groups, and the like.

It is a much better way to label the flakes than to label the cells themselves, as such labelling may detrimentally affect cell organelles including the nucleus and the nucleic acids, as well as cell physiology, cell enzymes, cell metabolism and the like.

Such labelling does for example allow the design of tissues consisting of more than one cell type, as the respective flakes can be recognized, selected and positioned in a scaffold, for example, by means of their labelling. Such positioning can also be done automatically, e.g. by a dedicated robot, in which case the respective labels or markers (e.g. fluorescent markers) are detected automatically by the robot.

The labelling does moreover allow the cell-type specific application of growth factors, which is very helpful in the engineering of tissues which comprise different cell types. Again, a pipetting robot may be used for this purpose.

When being used in cell therapy, the layer that is not contacting the cells may be labelled with tissue specific antigens. Thus, the delivery of the flakes and their content to the target site is supported.

In this context, X-ray markers and MRI-markers can also facilitate the targeting of the rolled flakes. For this purpose, an X-ray-tomograph or an MRI-tomograph can be used. Moreover, the respective labels could be used to control the wrapping and unwrapping of the flakes inside the target organism, or their integrity or degradation, respectively.

This approach helps to reduce the number of cells needed in cell therapy, for example, which results in a reduction of costs and resources, as cultured cells are expensive, and their production is time- and labour intensive. More important, the number of cells which do not remain in the area of therapy is reduced. This again reduces any unwanted side effects of cells which float freely in the target body, and may thus cause cancer or other diseases.

Furthermore it is preferred that the flakes comprise agents which enhance the biocompatibility. For example, the layer that is not contacting the cells may comprise an anticoagulant, e.g. heparin moieties, to avoid blood clotting. The person skilled in the art will select other agents which enhance the biocompatibility according to the specific needs. For example, it is possible to modify the layer contacting the cells with specific anchoring molecules, growth factors and the like.

It is particularly preferred that the flakes according to the invention comprise a material consisting of a hydrogel.

The term “hydrogel” as used herein implies that at least a part of the respective material comprises polymers that in water form a water-swollen network and/or a network of polymer chains that are water-soluble. Preferably the hydrogel permeation layer comprises in swollen state ≧50% water and/or solvent, more preferably ≧70% and most preferred ≧80%, whereby preferred solvents include organic solvents, preferably organic polar solvents and most preferred alkanols such as Ethanol, Methanol and/or (Iso-) Propanol.

A responsive hydrogel is particularly preferred. In the sense of the present invention, the term “responsive” means and/or includes especially that the hydrogel is responsive in such a way that it undergoes a change of shape and total volume upon a change of a specific parameter, like the addition of a target molecule or the application of a specific stimulus, the nature of which is further specified above (e.g. induced change of pH, induced change of temperature, induced exposure to electromagnetic waves, induced exposure to specific salts or organic compounds, or to a given concentration thereof, application of an electric field, application of a magnetic field, application of sound, application of vibrations), Other stimuli include the presence or concentration of dedicated analytes such as enzymes or other biomolecules. (see comment above).

Hydrogels are known to be shape sensitive to pH, ion concentration, temperature, solvent composition and electric potential. These parameters may cause a change in phase, shape, mechanics, refractive index, recognition or permeation rates that subsequently can be reversed to return the material to its original state. Stimuli-sensitive hydrogels have also been integrated with enzymes, protein mimics, and antibodies to design controllable actuators. These hydrogels have been shown to swell, or shrink, upon addition of a target molecule. The amount of swelling (or shrinking) of these hydrogels was attributed to changes in non-covalent interactions within the polymer network. The hydrogels can be also designed to swell, or shrink, upon presence of a target molecule; even they can be constructed in a way that the magnitude of swelling (or shrinking) can be proportional to the concentration of ligand present.

According to an embodiment of the present invention, the hydrogel material comprises a material selected out of the group comprising poly(meth)acrylic materials, substituted vinyl materials or mixtures thereof, as well as include epoxydes, oxetanes, and thiolenes.

According to another embodiment of the present invention, the hydrogel material comprises a poly(meth)acrylic material made out of the polymerization of at least one (meth)acrylic monomer and at least one polyfunctional (meth)acrylic monomer.

According to yet another embodiment of the present invention, the (meth)acrylic monomer is chosen out of the group comprising (meth)acrylamide, hydroxyethyl(meth)acrylate, ethoxyethoxyethyl(meth)acrylate or mixtures thereof.

According to still another embodiment of the present invention, the polyfunctional (meth)acrylic monomer is a bis-(meth)acryl and/or a tri-(meth)acryl and/or a tetra-(meth)acryl and/or a penta-(meth)acryl monomer.

According to an embodiment of the present invention, the polyfunctional (meth)acrylic monomer is chosen out of the group comprising bis(meth)acrylamide, diethyleneglycoldi(meth)acrylate, triethyleneglycoldi(meth)acrylate, tertraethyleneglycoldi(meth)acrylatetripropyleneglycoldi(meth)acrylates, pentaerythritol tri(meth)acrylate polyethyleneglycoldi(meth)acrylate, ethoxylated bisphenol-A-di(meth)acrylate, hexanedioldi(meth)acrylate or mixtures thereof.

Other materials which turned out in tests carried out by the inventors to be suitable for the above purposes include Ethylhexyl acrylate, Hydroxyethyl methacrylate, PNIPAA-co-isobutylmethacrylate (80:20), PMMA, PMMA-co-trimethylolpropane triacrylate, TMPTA, DEGDA, DEGDMA, Polystyrene, PMMA-co-DEGDA (2:1), PMMA-co-DEGDMA (2:1), PS-co-TMPTA (2:1), PS-co-DEGDA (2:1), PS-co-DEGDMA (2:1) and tris 2-hydroxyethyl isocyanurate triacrylate.

According to an embodiment of the present invention, the hydrogel material comprises an anionic poly(meth)acrylic material, preferably selected out of the group comprising (meth)acrylic acids, arylsulfonic acids, especially styrenesulfonic acid, itaconic acid, crotonic acid, sulfonamides or mixtures thereof, and/or a cationic poly(meth)acrylic material, preferably selected out of the group comprising vinyl pyridine, vinyl imidazole, aminoethyl (meth)acrylates or mixtures thereof, co-polymerized with at least one monomer selected out of the group neutral monomers, preferably selected out of the group vinyl acetate, hydroxyethyl (meth)acrylate (meth)acrylamide, ethoxyethoxyethyl(meth)acrylate or mixture thereof, or mixtures thereof.

It is known for a wide range of these co-polymers to change their shape as a function of pH or temperature, and to respond to an applied electrical field and/or current. Therefore these materials may be of use for a wide range of applications within the present invention.

According to an embodiment of the present invention, the hydrogel material comprises a substituted vinyl material, preferably vinylcaprolactam and/or substituted vinylcaprolactam.

According to an embodiment of the present invention, the hydrogel material is based on thermo-responsive monomers selected out of the group comprising N-isopropylamide, diethylacrylamide, carboxylsopropylacrylamide, hydroxymethylpropylmethacrylamide, acryloylalkylpiperazine and copolymers thereof with monomers selected out of the group hydrophilic monomers, comprising hydroxyethyl(meth)acrylate, (meth)acrylic acid, acrylamide, polyethyleneglycol(meth)acrylate or mixtures thereof, and/or co-polymerized with monomers selected out of the group hydrophobic monomers, comprising (iso)butyl(meth)acrylate, methylmethacrylate, isobornyl(meth)acrylate or mixtures thereof. These co-polymers are known to be thermo-responsive and therefore may be of use for a wide range of applications within the present invention.

A preferred example for these responsive hydrogels is Poly-n-isopropylacrylamide (PNIPAA).

The above mentioned hydrogels are highly permeable for low-molecular compounds, i.e. salts, sugars, lipids, growth factors, oxygen, and the like. For this reason, flakes comprising these gels will provide appropriate growing conditions for cells, even in the wrapped state.

In another embodiment, the layer which is in contact with the cell is not an hydrogel material. In these cases, it is preferred that the contact layer is structured such that it contains holes or micropores to allow the transport of nutrients and metabolites. However, transport can also take place trough the “open sides” of the wrap.

Furthermore, it is preferred that the flakes comprise a bilayer structure. By selecting suitable materials for the different layers, a different swelling of the two layers can be accomplished upon a given stimulus, e.g. due to different thermal expansion coefficients, water uptake or the like. This difference provides the driving force needed for the movement, i.e. the curling, of the flakes, which results in the rolled state. In a preferred embodiment of the former, the flakes consist of a non-responsive layer and a layer made from a responsive hydrogel.

Flakes can also comprise a tri- or multilayer structure or a gradient structure, i.e. a vertical concentration gradient of swelling or gelling components, which may result in similar behaviour. The above mentioned gradient can be produced with methods well know for the person skilled in the art. A gradient mixer, as commonly used to produce gradient gels for electrophoresis, can for example be used for this purpose. Another approach to create composition gradients is to induce the latter by use of a vertical gradient in polymerisation rate (e.g. intensity gradient by using a UV absorber).

In this context, it is worth mentioning that at least one of the above mentioned layers can comprise a structured surface.

A tri-layered embodiment can for example comprise a top layer which is very thin, thus not affecting the stress mechanics which lead to the rolling movement, but which comprises a composition that is well compatible with, or even promotes, cell growth.

Yet, in cases where adherent cell sheets with high confluency are grown on the flakes it is as well possible to use single layer flakes, i.e. flakes without a gradient or a stratified arrangement. In these cases the cell sheet itself acts as the second layer and the differences in expansion behaviour between the single layered flakes and the adherent cell sheet results in the rolling-up of the flakes into the wrapped state. This feature is especially useful for another preferred embodiment of the present invention, wherein the adhesion of cells is used to determine in which cell cycle state the cells are, as in some states a cell applies more tensile forces to its adhesion points than in other states.

In yet another preferred embodiment it is provided that the flakes comprise a structured cell contacting surface. This may stimulate cell adhesion, or help to specifically direct the growth and orientation of the cells. In these cases, a structured cell contact layer or a structured non contacting layer can induce a preferred rolling direction of the flake.

It is moreover preferred that during cell culturing, external stimuli are being applied in order to influence cell growth or cell differentiation.

Such stimuli can for example be selected from the group consisting of the application of growth factors, application of mechanical stress, or the application of electric or magnetic fields. In particular, these stimuli can enforce or prevent cell differentiation, according to cell types and to the respective application, as well as the direction and shape of cell growth can be controlled.

Growth factors can also be added to the layer that is in contact with the cell in order to provide that cell growth is still stimulated once the cells have been wrapped.

Furthermore, it is preferred that the flakes are disposed or produced on a support structure before cells are seeded.

In a preferred embodiment the flakes comprise a material which is biodegradable and/or biologically safe. This is an important feature both for tissue engineering and for use of the flakes in cell therapy. For example, the use of biodegradable material allows, under certain circumstances, that the flakes are directly administered to a subject. Likewise, said material, when the flakes are being used for in vitro tissue engineering, can be selected as to slowly disintegrate, wherein the speed of disintegration corresponds to the speed of cell growth and of the production of extra cellular matrix by the growing cells. In this embodiment, the extracellular gel matrix is stepwise replaced by living cell matter.

Suitable biodegradable materials are for example described by Gunatillake P and Adhikari R, European Cells and Materials (5) 2003, 1-16, the whole content of which is herein incorporated by reference. Among these are Poly(glycolic acid) (PGA), poly(lactic acid) (PLA) and their copolymers and derivatives, like Poly(d,l-lactic-co-glycolic acid), Polylactones like Poly(caprolactone) (PCL) and their derivatives, Poly(propylene fumarate) (PPF) and its derivatives, Polyanhydrides like Poly [1,6-bis(carboxyphenoxy)hexane], Tyrosine-derived polycarbonates and their derivatives, Polyorthoesters (POE) and their derivatives, Polyurethanes (PU) with non-toxic degradation products like lysine diisocyanate (LDI, 2,6-diisocyanatohexanoate) and other aliphatic diisocyanates like hexamethylene diisocyanate (HDI) and 1,4-butanediisocyanate, e.g. Poly(glycolide-co-γ-caprolactone), Polyphosphazenes like Ethylglycinate Polyphosphazene and their derivatives, and so forth.

Other biodegradable polymers comprise poly(maleic acid), poly(p-dioxanone), poly(trimethylen-carbonate), poly(3-hydroxibutarate), poly(3-hydroxyvalorate and their copolymers. A class of suitable responsive biodegradable polymers is Poly(N-(2-hydroxypropyl) methacrylamide mono/dilactate), as described by Soga O et al, Biomacromolecules 2004 (5) 818-821, the whole content of which is herein incorporated by reference.

Other suitable biodegradable materials comprise alginate, hyaluronic acid, chitosan, collagen, gelatin, silk or combinations thereof.

These biodegradable materials can be used by themselves, or they are being used in a network together with crosslinking agents. The created network will disintegrate after some time, and, given the crosslinking agents have small molecular weights, the latter will be washed out. In another embodiment, the biodegradable material is immobilized in a network that consists of non-biodegradable matter.

In another preferred embodiment it is provided that the flakes comprise a material which disintegrates upon application of an extrinsic stimulus. This feature is for example useful in cell therapy, in order to enhance the release of the cells at the target site. This can for example be accomplished by application of ultrasound or infrared light, as both exhibit good penetration into the human or animal body.

For this purpose, the rolled flakes are injected and targeted via targeting moieties on the outside of the rolled flakes, and the latter can un-roll upon application of an external stimulus and thus exposing the cells to the area of interest. Alternatively, the flakes are not targeted but homogeneously distributed over the body, while the external stimulus is focussed only on the area of interest. Only there the rolled flakes will unroll and release their cellular content.

Preferably, the cells used in the procedure according to the invention are adhering cells. However, suspended cells may also be used. In these cases, it can be provided that the liquid comprising the suspended cells remains in the rolled flakes due to capillary forces.

A preferred application of the flakes produced with the above process comprises that after transferring the flakes from the planar state into the rolled state, the rolled flakes are being distributed in a predefined spatial pattern in order to produce a three dimensional tissue. This approach may likewise be used for tissue engineering in vitro and in vivo. In order to achieve this aim, a scaffold may be used which predetermines the shape of the tissue, or organ, respectively, that is to be produced, and in which the cell wraps according to the invention are deposited.

With this approach it is possible to obtain a heterogeneous distribution of different cell types which have been cultured and specifically treated in different cell cultures and subsequently wrapped in different flakes). In this way it is possible to precisely control which cell type is where in the newly grown tissue. Alternatively, one single omni- or pluripotent cell type (stem cell or progenitor cell) can be cultured on different flakes containing different factors stimulating the differentiation into different cell types. The wraps can then be distributed in a predefined matter and during tissue engineering the cells will mature into different tissue types. Thus, a bone-cartilage interface construct can for example be obtained.

Another preferred application of the flakes provides that after transferring the flakes from the planar state into the rolled state, the rolled flakes are being aligned with their open ends to one another. In this way, vessels or continuous neurons can be formed. Such alignment of the rolled flakes can be stimulated by external factors such as geometrical restraints, external magnetic or electric fields, flow and shear, and the like.

The same type of vessel-like tissues can however be achieved when flakes are being used which, are transfer, adopt a helix like shape, as set forth above.

Still another preferred application of the flakes provides that different flakes bearing grown cells are superimposed before transferring the flakes from the planar state into the rolled state. By transferring the superimposed flakes into the rolled state, one achieves a coaxial arrangement of cells, which may have different origin. This enables the production of tubular tissues, like blood vessels, with a stratified arrangement of different cells, like endothelium, connective tissue, vascular smooth muscle cells and the adventitia (containing nerves).

The same result can however be achieved by depositing and aligning rolled-up flakes onto substrates with patterned planar flakes bearing cells. Upon application of the above mentioned stimulus, these flakes will transfer from the planar state into the rolled state flakes, thus wrapping around the deposited, rolled-up flakes. Again, the same type of vessel-like tissues can be achieved when flakes are being used which, are transfer, adopt a helix like shape, as set forth above.

Another preferred application of the flakes provides that the rolled flakes are deep-frozen. By this means, they can be stored for future use, the latter being specified elsewhere in this specification.

In yet another preferred embodiments of the present invention it is provided that the cell wraps and/or the assemblies of cell microcarriers thus produced are delivered to
i) an in vitro tissue- and/or organ-engineering environment, and/or
ii) a damaged tissue and/or organ of a human or an animal.

The term “in vitro tissue- an/or organ-engineering environment”, as used herein, shall refer to a culture environment in which the cells are supposed to proliferate in order to form a tissue and/or an organ, or at least one or more parts thereof.

Such culture environment may comprise a suspension cell culture system, a two-dimensional cell culture system and/or a three-dimensional cell culture system, like solid porous scaffolds or matrices, which for example comprise biodegradable matter and/or collagen.

In the latter embodiment, stem cells, which help restore the tissue function of the damaged tissue, are preferably used. Such approach is very promising for the treatment of neurodegenerative diseases, like Alzheimer's or Parkinson's disease, as well as for the repair of necrotic tissues, as results from cardiac stroke, for example.

Here again, the cell wraps according to the invention provide a means for targeting the cells directly to the site of damage. This results in a reduction of costs and resources, and, moreover, the number of cells which do not remain in the area of therapy, and which eventually may cause cancer or the like, is reduced. The binding agents may, in this embodiment, be selected in such way that they are complementary to a tissue specific agent.

In both cases, the binding agents according to the invention provide a useful tool for a site specific targeting of the cell wraps, and for a controlled coupling of different cell wraps to one another, with the option to provide a self assembly process.

Furthermore, another preferred embodiment provides that the flakes comprising cells are administered orally to a subject, i.e. they are swallowed. The composition of the flakes can be selected to be resistant to saliva, gastric fluids and enzymes and/or to the enzymes of the small intestine, the pancreas or the gallbladder. By this means, it can be accomplished that the flakes remain intact in order to protect the cells until they reach predefined locations in the intestines, which is supported by the said binding agents in a manner described above. There the flakes could unroll, or disintegrate, respectively, due to the local pH or the presence of particular agents, e.g. enzymes, and let the cells do their work.

Still another preferred embodiment comprises that the rolled flakes are being implanted in a subject, either by injection or surgically, in order to take over endocrinic functions. In this case, the flakes contain endocrinic cells, i.e. pancreas cells, or cells producing endocrinic agents, like insulin. Targeting of the flakes is possible, as set forth above, by means of the said binding agents.

Due to the permeability of the flakes nutrients enter the lumen and thus feed the cells. Likewise, endocrinic agents can be secreted. At the same time, T-cells and macrophages are kept outside, thus preventing an immune reaction.

Furthermore, a process for the manufacture of a two-dimensional object (“flake”) and/or a cell wrap according to any of the aforementioned claims is provided, which process comprises the steps of:
a) providing a mould comprising a grid which creates wells defining the shape of the flakes;
b) casting a precursor material into the mould which, upon application of an extrinsic stimulus, is transferred from the planar state into a rolled state;
c) curing the cast material to obtain flakes;
d) optionally, transferring the flakes from the planar state into a rolled state (“cell wrap”) by application of said extrinsic stimulus
e) coupling at least one type of binding agent to the flakes before or after any of steps a)-c).

It is preferred that after curing the flakes are released from the mould and then used according to the above described processes. However, flakes can also remain in the mould. In this embodiment, the mould may for example serve as a support structure for subsequent cell culture.

It can be provided that this process is used to provide bi-, tri- or multilayered flakes. In this case, the casting and curing steps are to be repeated. By using different precursor materials for the different layers, it can be achieved that, as set forth above, the resulting flakes are transferable from a planar state into a rolled state by application of an extrinsic stimulus. Suitable materials are described in detail above. The curing can for example be accomplished by application of UV light, electron rays, heat or dedicated curing agents. However, self curing material can also be used.

In a preferred embodiment of this process it is provided that several layers are cast into the mould in order to obtain multi-layered flakes. This means that a first layer is first cast and cured. Thereafter a second layer is cast and cured, and so on. Likewise, it may be provided that throughout casting, a gradient structure is accomplished in the flakes, e.g. a vertical concentration gradient of swelling or gelling components. The gradient can for example be formed by applying a gradient in light intensity over the layer during curing.

Furthermore, it may be provided that any of the following additives is added to the precursor material:
(i) agents which enhance the biocompatibility;
(ii) biodegradable and/or biologically safe material;
(iii) labels or markers; and/or
(iv) growth factors;
(v) antibiotics.

In yet another preferred embodiment, it is provided that before or after curing a structured surface is accomplished in the flakes. This may for example be done by using a structured template which is pressed on the surface of the flakes. It is also possible to photopolymerize said hydrogel layer, e.g. with an absorber to get a gradient in thickness direction. In this context, a double (or multiple) UV exposure can for example be used. Preferably, one of the steps can comprise the use a mask in order to achieve a surface pattern.

Another option is to use polarized light in order to create a structured surface. In this case, a linearized structure can by obtained by selective curing, or photolysation, in the plane of polarization. By repeating this process with different planes of polarization, a grid pattern or the like can be created on the surface.

According to another aspect of the invention, a two-dimensional object (“flake”) is provided, having structural and/or material properties as set forth in the above process, or made with the above manufacturing process.

The aforementioned components, as well as the claimed components and the components to be used in accordance with the invention in the described embodiments, are not subject to any special exceptions with respect to their size, shape, material selection and technical concept such that the selection criteria known in the pertinent field can be applied without limitations.

Further objects of the present invention are
a two-dimensional object (“flake”) and/or a cell microcarrier (“cell wrap”) used according to the invention, or made with a process according to the invention.
an assembly of cell microcarriers (“cell wraps”) as manufactured with a process according to the invention, and/or
an assembly of cell microcarriers (“cell wraps”) comprising cell microcarriers made with a process according to the invention
an assembly of cell microcarriers (“cell wraps”) consisting of two-dimensional objects bearing cells and transferred into the rolled state by application of an extrinsic stimulus, wherein at least two cell microcarriers are bound to one another by means of at least one binding agent.

### BRIEF DESCRIPTION OF THE DRAWINGS

Additional details, features, characteristics and advantages of the object of the invention are disclosed in the subclaims, the figures and the following description of the respective figure and examples, which, in an exemplary fashion, show preferred embodiments of a process and flakes according to the invention.

FIG. 1 shows the first steps of a process according to the invention;

FIG. 2 shows the succeeding steps of a process according to the invention;

FIG. 3 shows a photomicrograph of two cell wraps according to the invention, comprising a bilayer structure containing a responsive hydrogel material;

FIG. 4 shows the principle of coupling binding agents to the flakes;

FIG. 5 shows how the binding agents can be used to bind the said cell-cell wraps to other entities;

FIG. 6 demonstrates the formation of a cell assembly based on cell wraps containing different areas with different binding properties;

FIG. 7 illustrates a process in which a given cell wrap type comprises a matrix-specific binding agent;

FIG. 8 shows an embodiment of the invention, in which a multiplicity of different binding agents being used;

FIG. 9 shows an embodiment of the present invention in which cell wraps attach only temporarily to other cell wraps; and

FIG. 10 shows an embodiment in which a cell wrap is attached to another cell wrap and later again removed by cleaving

### DETAILED DESCRIPTION OF EMBODIMENTS

In the following, the present invention is demonstrated by means of examples, which by no means should be understood as to limit the scope of the invention.

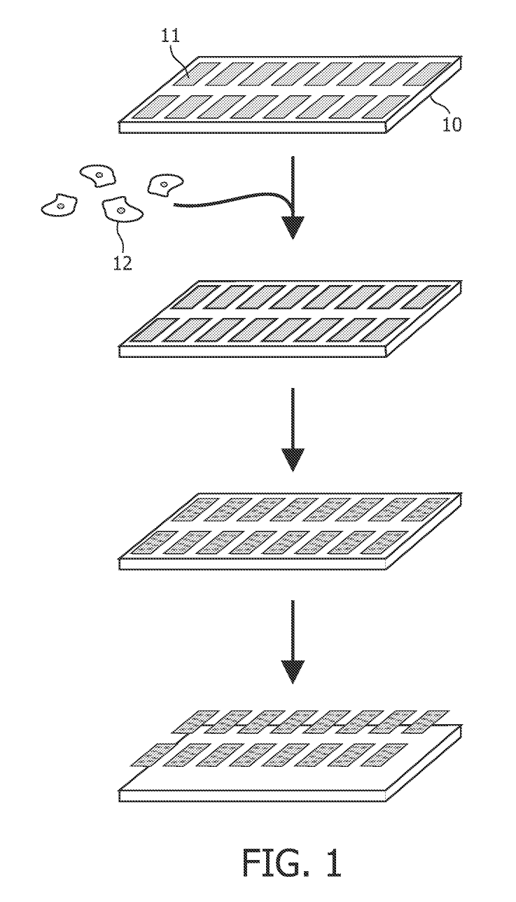

FIG. 1 shows the first steps of a process according to the invention. Therein, a support structure 10 is provided upon which two-dimensional objects 11 having two sides (“flakes”) 1 are being disposed or produced.

The said flakes comprise a material which, upon application of an extrinsic stimulus, is transferred from the planar state into a rolled state. Cells 12 are then seeded on one side of said flakes (“cell-bearing side”), and the substrate is kept under conditions that enable cell growth. Before the flakes are further processed, they are released from the support structure. Typical dimensions of the flakes are in the order of the dimensions of the cells or somewhat bigger than that, e.g. 100×100 μm.

It is obvious from FIG. 1 that the cells are only present on the flakes and not on the substrate in between the flakes. In order to avoid that cells settle down in the interstitium between the flakes, the surface of the substrate underneath can be modified such that cells do not adhere to it, or that the cells have a clear preference to grow on the flakes rather than on the substrate. However, in some cases it does not matter if cells also grow on the substrate. As soon as the flakes are released and rolled up, the substrate with left-over cells can be discarded.

In some special cases not shown here it can even be beneficial to have cells in the interstitium between the flakes. For instance, some difficult cell-types need co-feeder cells for growth, namely for the production of the appropriate growth factors. These feeder cells can be seeded in between the flakes, while the cells of interest are grown on the flakes.

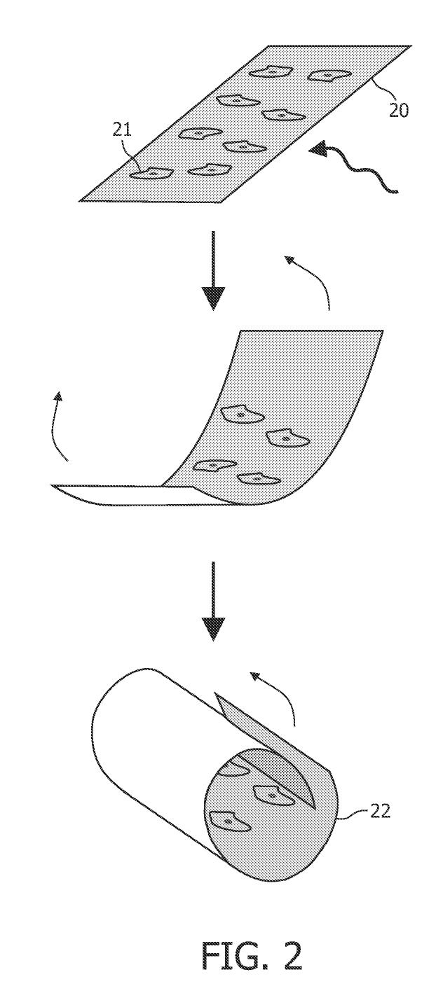

FIG. 2 shows the succeeding steps of a process according to the invention. A flake 20 with cells 21 is exposed to an extrinsic stimulus, symbolized by the undulated arrow.

Said stimulus may consist of heat, a pH-change, or exposure to electromagnetic waves, for example (see specification for further details). The exposure to said stimulus elicits a response in the flakes, namely that the former are being transferred from a planar state into a rolled state (“cell wrap”), as indicated by the respective arrows. In this manner, cell wraps as defined above are being produced.

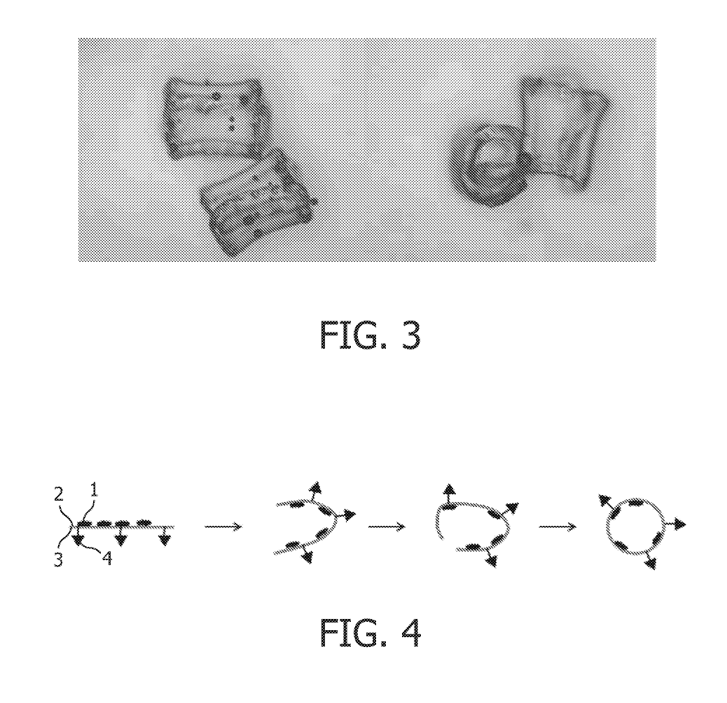

FIG. 3 shows a photomicrograph of two cell wraps according to the invention, comprising a bilayer structure containing a responsive hydrogel material. The cell wraps are 700×1400 μm in size, and rolled up they have a diameter of approximately 500 μm. The stimulus used to transfer the flakes from the planar state to the cell wrapped state was a thermal stimulus. To induce the cell wrapping movement, the flakes were placed in a water bath at room temperature. At room temperature the responsive hydrogel shows strong swelling in water and as a result the patterned flakes were released from the substrate and rolled. Upon heating the water bath above 33° C., the hydrogel collapses (PNIPAA has a lower critical solution temperature (LCST) at 33° C.) and the hydrogel layer shrinks. As a result, the bi-layers unroll upon heating the water bath above 33° C.

FIG. 4 shows the principle of coupling binding agents to the flakes, which—in this example, consist of the two layers 2 and 3. For the sake of simplicity, the flakes and cell wraps shown in this application are only depicted with one or two layers, although they may as well have three or more layers.

Cells 1 grow on the upper layer 2 of the flakes. The said binding agents 4 are symbolized as arrows which have been coupled to the lower layer 3 of the flakes, i.e. to the side which is opposite to the cell-bearing side. Once the flakes are transferred into their rolled state (“cell wraps”) the said binding agents are disposed on the outside of these cell wraps, and can then be used to bind the cell wrap thus achieved to at least one other entity, as described above.

It is yet to be mentioned that, as a variation of the process shown in FIG. 4, the binding agents can be coupled to the flakes after they have already been transferred into their rolled state. Examples for binding agents and their complementary counterparts are given in table 1.

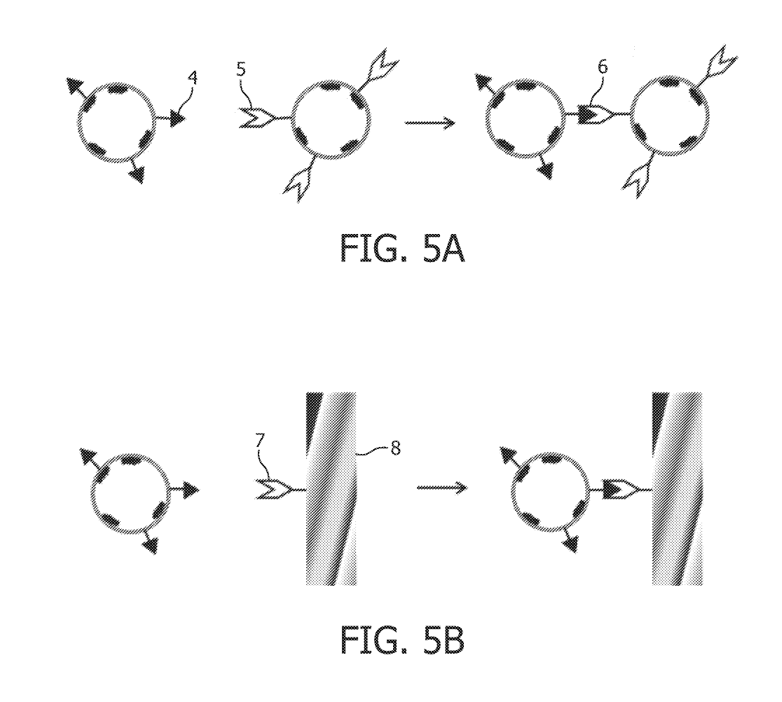

FIG. 5 shows how the binding agents can be used to bind the said cell-cell wraps to other entities. According to FIG. 5A cell wraps with different binding agents 4 and 5 which are complementary are used to bind two different, or similar cell wraps to each under formation of a covalent or non-covalent bond 6, which is specific and has a high affinity.

According to FIG. 5B, the said binding agent can also be used to bind a cell wrap to entities 8 different than cell-cell wraps, such as organs, tissues, artificial scaffolds and the like. The complementary binding agent 7 can e.g. be used to target the cell wrap to a specific tissue 8. For this purpose, the said complementary binding agent 7 may for example be a tissue-specific marker complementary to the cell wrap's binding agent.

Likewise, the entity 8 may as well be part of a three dimensional matrix, like solid porous scaffolds which are being used for in vitro tissue engineering, for example comprising biodegradable matter and/or collagen.

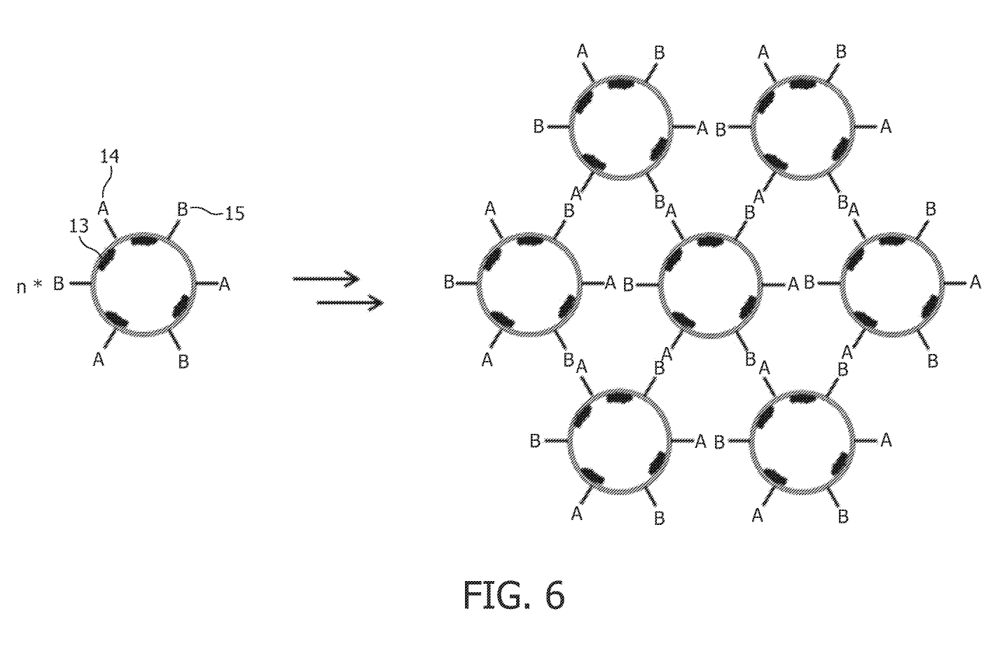

FIG. 6 demonstrates the formation of a cell assembly based on cell wraps containing different areas with different binding properties. The outer layer of the cell wraps in this example consists, in this example, of six alternating areas, out of which three 3 comprise a binding agent A (14) and three comprise a binding agent B (15). It is important, in this example, that all cell wraps used have the same pattern of binding areas. The binding agents A and B will then bind to each other covalently or non-covalently, depending on their chemical nature, and for thus a network of cell wraps can be formed. It is important that, under certain circumstances, the binding process will take place without any additional steps, i.e. it is thus a self assembly process.

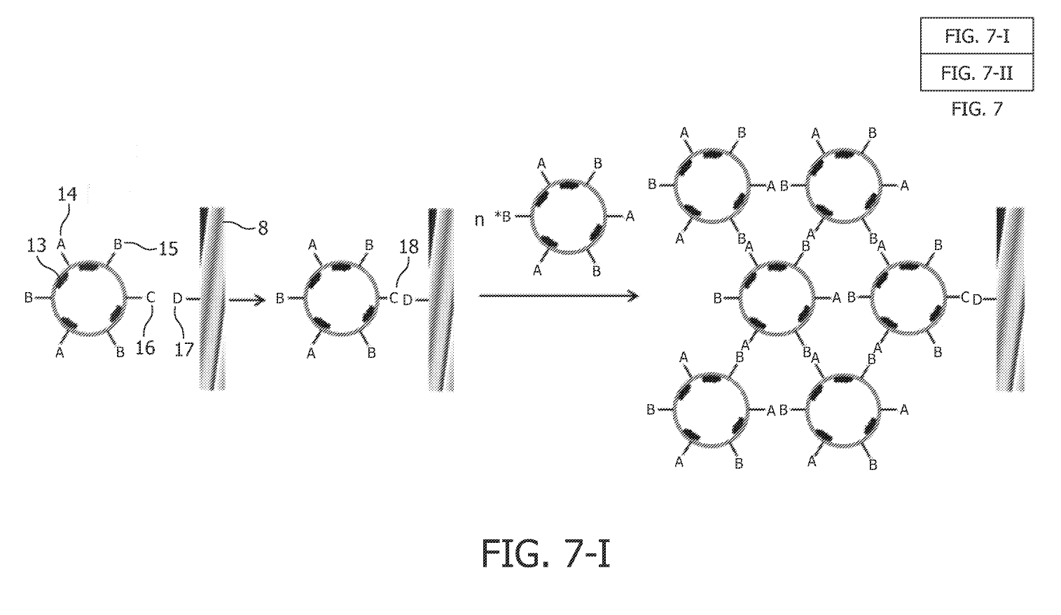

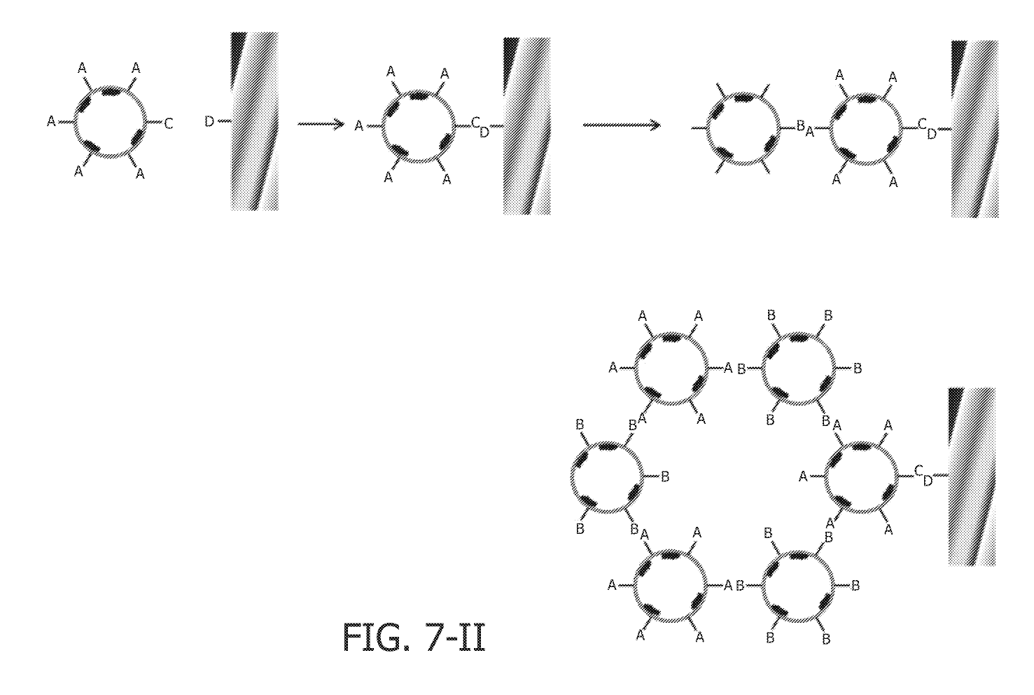

FIG. 7 illustrates a process in which a given cell wrap type comprises, despite the earlier described binding agents A (14) and B (15), a matrix-specific binding agent C (“homing molecule”; 16) for binding the cell wrap to a three-dimensional matrix 8 (e.g. human organs, tissues, scaffold, etc.) carrying a complementary binding agent D (17). Once the cell wrap has bound to the matrix, other cell wraps carrying binding agents complementary to A and B m are then bound to the first cell wrap, which in this embodiment double-acts as an anchoring device. In such manner, a structured artificial tissue can be designed. The binding agent C can therefore be used to guide a cell wrap specifically to a target tissue in the body whereas afterwards the binding agents A and B can be used to build up the new artificial tissue in a structured way.

While in FIG. 7A, the cell wraps bound to the first cell wrap (the “anchoring device”) are identical, FIG. 7B shows a modification of the said process in which the former are non-identical. For example, the cell wrap shown on the left carries only binding agents of type B. These different cell wraps allow for the production of more complex tissues, while under certain circumstances the process can still be a self-assembly process.

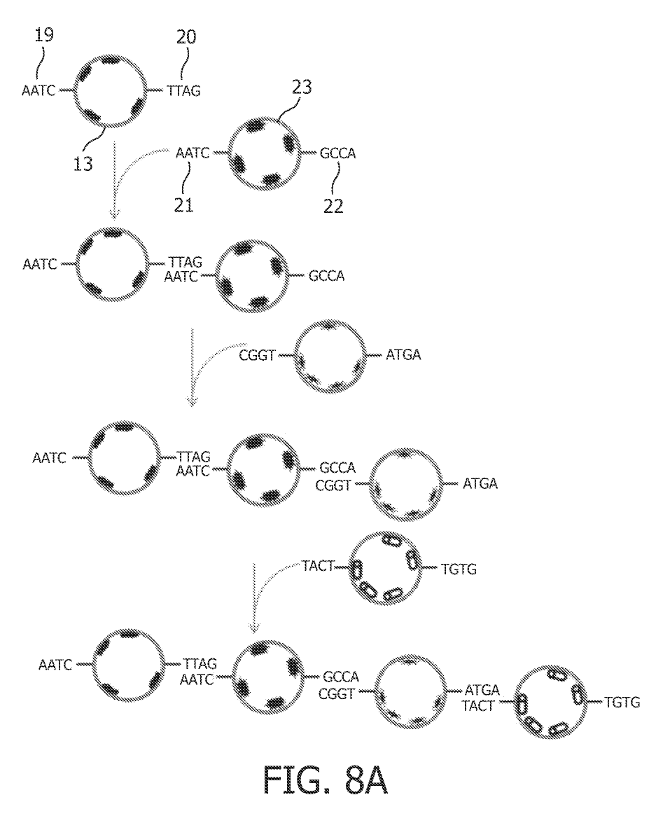

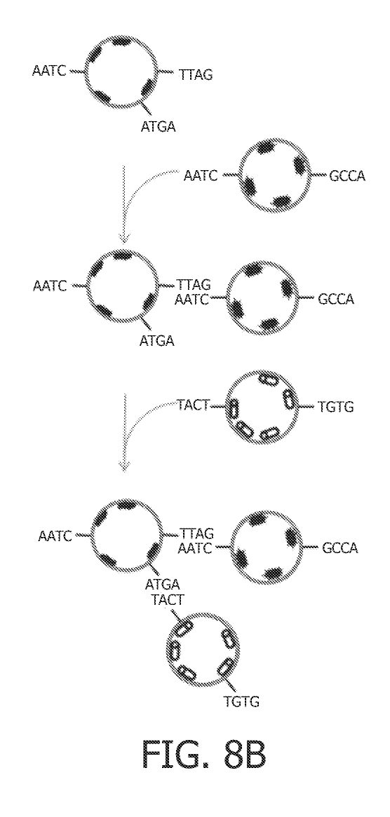

FIG. 8A shows another embodiment of the invention, in which a multiplicity of different binding agents 19-22 is being used to bind a manifold of different cell wraps 13, 23 containing each a certain cell type. In this embodiment, the binding agents are oligonucleotides, where cell wrap 13 is binding to cell wrap 23, which may for example contain different cells than the former. The oligonucleotide binding agent 20 (TTAG) is complimentary to the oligonucleotide binding agent 21 (AATC), while the oligonucleotide binding agent 22 (GCCA) is complimentary to the oligonucleotide binding agent of the following cell wrap, and so forth.

The oligonucleotide binding agents of FIG. 8A comprise of single-stranded quadramers, although the said oligonucleotides can of course be longer, and/or be double-stranded. (see above).

As oligonucleotides bind to one another with high specifity (according to their sequence), a high diversity in binding combinations can thus be achieved. An oligonucleotide comprising 4 nucleotides can thus be produced in 4⁴=256 variations, while an oligonucleotide having 6 nucleotides can be produced in 4⁶=4096 variations. This means that the longer the oligonucleotide is, the higher the diversity of binding agents, and the higher the binding strength and specifity is.

FIG. 8B shows a combination of the principles of highly specific binding of different cell wraps and the use of different binding molecules in one cell wrap. In such way it is possible to build complex networks of cell wraps allowing to bring several cell types into specific location within an artificial tissue to be built.

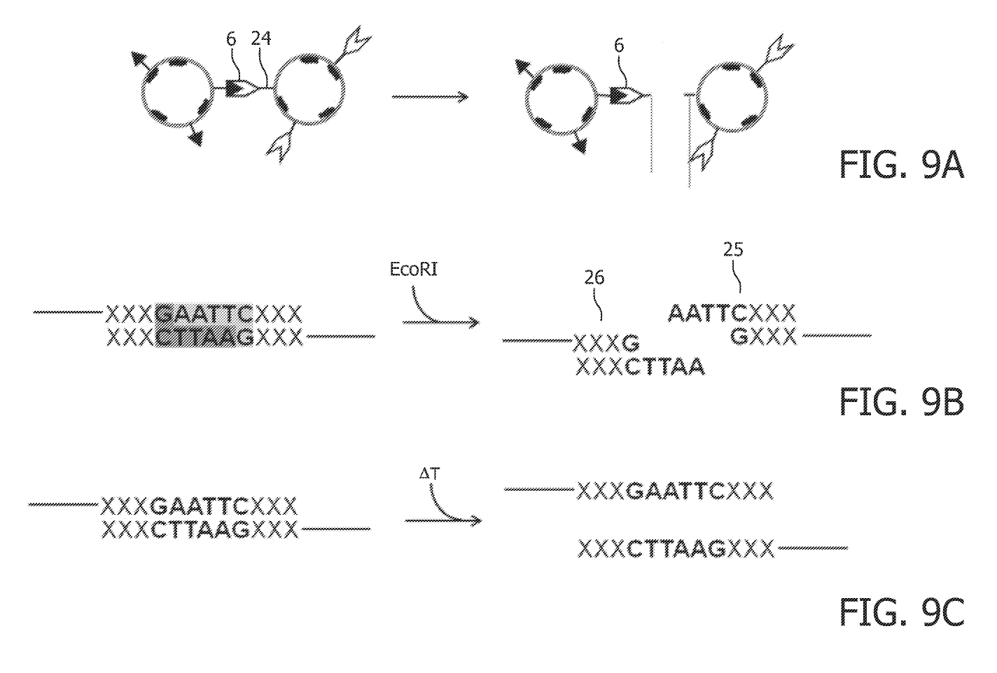

FIG. 9A shows another embodiment of the present invention. Here, cell wraps attach only temporarily to other cell wraps. This can be achieved by either cleaving the specific bond 6, or by cleaving a particular linker 24, which as been incorporated into one of the binding agents just for this purpose. By the cleaving process, the said linker is then separated in two parts 25 and 26.

In order to achieve this, the linker can
(i) be a peptide that can be specifically hydrolysed by a peptidase or protease, or
(ii) the linker can consist of an oligonucleotide that can be cut specifically by a restriction endonuclease, like EcoRI, as depicted in FIG. 9B, or the linker can consist of an oligonucleotide that upon heating (see above, particular under help of local heating procedures and/or agents lowering the melting temperature) melts, as depicted in FIG. 9C.

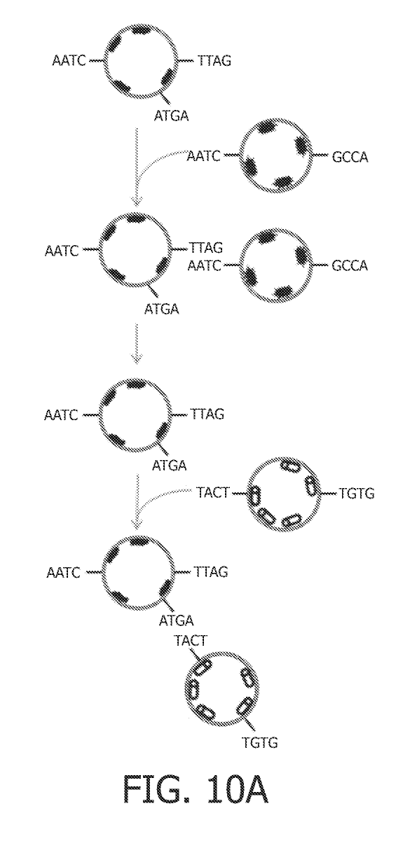

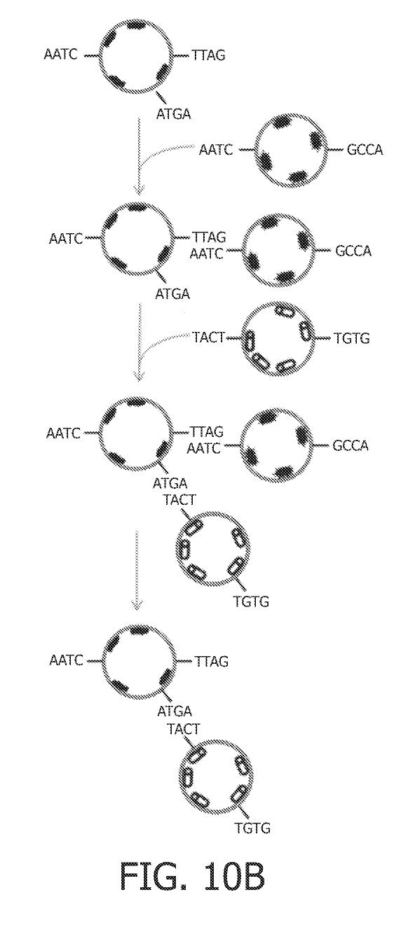

The possibility to remove cell wraps in the course of the process allows for the sequential binding and removal of different cell wraps, as depicted in FIG. 10.

In FIG. 10A it is shown that a cell wrap is attached to another cell wrap and later again removed by cleaving, then another cell wrap is added. According to FIG. 10B one kind of cell wrap is added, subsequently another kind is added and then the first cell wrap is removed by cleaving. The said process can be varied and continued, at least in theory, indefinitely.

1. A process for providing an assembly of cell microcarriers (“cell wraps”), comprising the steps of
a) providing planar, two-dimensional objects having two sides (“flakes”), wherein these objects comprise a material which, upon application of an extrinsic stimulus, is transferred from the planar state into a rolled state,
b) providing cells on one side of said flakes (“cell-bearing side”),
c) transferring the flakes from the planar state into a rolled state (“cell wrap”) by application of said extrinsic stimulus, and
d) coupling at least one type of binding agent to the flakes before or after any of steps a)-c).

2. The process according to claim 1, characterized in that said binding agents are coupled to the side of the flakes which is opposite to the cell-bearing side.

3. The process according to claim 1, characterized in that said process comprises the additional step of
e) binding at least one cell wrap thus achieved to at least one other entity by means of at least one of said binding agents.

4. The process according to claim 1, characterized in that said binding agents are selected from the group consisting of proteins and polypeptides, nucleic acids, molecular tags, ligands and/or charged groups and/or said binding agents are selected in such way that they confer, to the flakes, or to subsections of the former, hydrophobic and/or hydrophilic properties.

5. The process according to claim 1, characterized in that said binding agents are capable of building up covalent bonds.

6. The process according to claim 1, characterized in that at least two different types of binding agents are added to said flakes in a patterned fashion.

7. The process according to claim 1, characterized in that step b) comprises the substeps of
b1) seeding cells on said flakes, and
b2) growing said cells.

8. The process according to claim 1, characterized in that said stimulus is selected from the group consisting of
induced change of pH,
induced change of temperature,
induced exposure to electromagnetic waves,
induced exposure to ions, specific salts or organic compounds, or to a given concentration thereof,
application of an electric field,
application of a magnetic field,
application of sound,
application of vibrations,
induced exposure to, or an induced suppression of, enzymes and other biomolecules,
induced release from the substrate, and/or
induced exposure to a solvent composition.

9. The process according to claim 1, characterized in that the flakes comprise a material consisting of a hydrogel and/or the flakes comprise a bilayer structure, a trilayer structure, a multilayer structure and/or a gradient structure.

10. The process according to claim 1, characterized in that different flakes bearing grown cells are superimposed before transferring the flakes from the planar state into the rolled state.

11. The process according to claim 1, characterized in that the cell wraps and/or the assemblies of cell microcarriers thus produced are delivered to
i) an in vitro tissue- and/or organ-engineering environment, and/or
ii) a damaged tissue and/or organ of a human or an animal.

12. A process for the manufacture of a two-dimensional object (“flake”) and/or a cell wrap according to claim 1, comprising the steps of:
a) providing a mould comprising a grid which creates wells defining the shape of the flakes;
b) casting a precursor material into the mould which, upon application of an extrinsic stimulus, is transferred from the planar state into a rolled state;
c) curing the cast material to obtain flakes;
d) optionally, transferring the flakes from the planar state into a rolled state (“cell wrap”) by application of said extrinsic stimulus
e) coupling at least one type of binding agent to the flakes before or after any of steps a)-c).

13. A two-dimensional object (“flake”) and/or a cell microcarrier (“cell wrap”) used according to claim 1.

14. An assembly of cell microcarriers (“cell wraps”) as manufactured with a process of claim 1.

15. An assembly of cell microcarriers (“cell wraps”) comprising cell microcarriers made with a process according to claim 12, or consisting of two-dimensional objects bearing cells and transferred into the rolled state by application of an extrinsic stimulus, wherein at least two cell microcarriers are bound to one another by means of at least one binding agent.
